(19) United States
(12) Patent Application Publication
(10) Pub. No.: US 2015/0296111 A1
(43) Pub. Date: Oct. 15, 2015

(54) FINGER COT CAMERA SYSTEM

(71) Applicant: Mayo Foundation for Medical Education and Research, Rochester, MN (US)

(72) Inventors: Elizabeth Rajan, Rochester, MN (US); Kevin Lindeen, Rochester, MN (US)

(21) Appl. No.: 14/647,207

(22) PCT Filed: Nov. 25, 2013
(86) PCT No.: PCT/US2013/071713

§ 371 Date: May 26, 2015

Related U.S. Application Data
(60) Provisional application No. 61/729,863, filed on Nov. 26, 2012.

(51) Int. Cl. H04N 5/225; A61B 1/012; A61B 5/00; A61B 1/06; A61B 1/05; A61B 1/00

(52) U.S. Cl. CPC H04N 5/2257; A61B 1/053; A61B 1/00016; A61B 1/00147; A61B 5/6826; A61B 1/0676; A61B 1/0684; A61B 1/00032; A61B 1/012; H04N 2005/2255

(57) ABSTRACT

This document provides methods and devices for medical diagnostic procedures using a camera system. For example, devices having a camera coupled to a finger cot for use during digital exams of body cavities such as the rectum are provided.

### CROSS-REFERENCE TO RELATED APPLICATIONS

This application claims the benefit of U.S. Provisional Application Ser. No. 61/729,863, filed Nov. 26, 2012. The disclosure of that prior application is considered part of (and is incorporated by reference in) the disclosure of this application.

### BACKGROUND

1. Technical Field

This document relates to methods and devices for medical diagnostic procedures using a camera system. For example, this document relates to devices having a camera (e.g., a disposable miniature video camera) located on a finger cot for use during digital exams of body cavities such as the rectum, vagina, oropharynx, wounds, and the like.

2. Background Information

One in four colorectal cancers is in the rectum, and many are within an examiner's reach on digital rectal examination. At present, general rectal and vaginal exams are performed with digital palpitation without the ability to visualize the area of concern, or to further inspect an area of abnormality. Detection of early abnormalities reduces illness and saves health care costs.

### SUMMARY

This document provides methods and devices for medical diagnostic procedures. For example, this document provides finger cot devices having one or more cameras (e.g., one or more disposable miniature video cameras) located on or near the tip of a finger cot for use during digital exams of body cavities such as the rectum, vagina, oropharynx, wounds, and the like.

In general, one aspect of this document features a method for performing a digital exam on a mammal. The method comprises inserting a camera within a body cavity of the mammal. The camera is removably coupled to a finger of an operator performing the digital exam.

In some aspects, an insertable camera device for performing a visual examination within a body cavity of a mammal includes a camera attached to a finger cot. The camera device is configured to be wearable on a finger of an operator, and to be inserted within the cavity by the operator wearing the camera device.

In some aspects, a finger cot camera system for performing a visual examination within a body cavity of a mammal includes a camera attached to a finger cot and an external viewing system. The camera is configured to be wearable on a finger of an operator, and to be inserted within the cavity by the operator wearing the device. The external viewing system is configured to receive and display images from the camera.

In some implementations, the method, device, and system may be used for examination of a mammal that is a human.

Particular embodiments of the subject matter described in this document can be implemented to realize one or more of the following advantages. In some embodiments, a camera device coupled to a finger cot can provide images from within a body cavity that will allow for identification of early abnormalities, e.g., cancer, fissures, fistulas, hemorrhoids, polyps and the like, while providing an ability to differentiate abnormalities from a normal finding such as fecal matter. Such a device can be useful for general physical exams for the community or hospital based general practitioner to determine which patients will benefit from formal exams, e.g., colonoscopy or vaginoscopy. In some cases, the devices provided herein can provide an improved result over digital exams by enabling more distant and better visualization. In some embodiments, the camera device can be a single-use disposable item, thereby adding to the convenience of using the system.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a schematic diagram of a finger cot camera system in accordance with some embodiments provided herein.

FIG. 2 is a schematic diagram of a finger cot camera device in accordance with some embodiments provided herein.

FIG. 3 is a side view of a finger cot camera device in accordance with some embodiments provided herein.

Like reference numbers represent corresponding parts throughout.

### DETAILED DESCRIPTION

This document provides methods and devices for medical diagnostic procedures using a camera system (e.g., a video camera system). For example, this document relates to a disposable miniature still camera or video camera coupled at or near the tip of a finger cot for use during digital exams of body cavities such as the rectum, vagina, oropharynx, wounds, and the like.

A finger cot camera system in accordance with some embodiments provided herein can include the following devices and subsystems. In general, the system can include: (i) an insertable camera device (e.g., a video camera device) for temporary placement within a body cavity of a patient, and (ii) an external viewing system (e.g., a video viewing system) with a viewing monitor (e.g., a video viewing monitor) located outside of the patient's body.

The insertable camera device can include a contoured housing fixedly attached to a finger cot, or a finger of a surgical glove, to be worn by an operator. The housing can include a visually transparent tip portion. A miniature camera can be mounted inside the housing in an orientation under the transparent tip portion to allow the camera to view images exterior to the housing. A light source can be included in the housing. The light source can be oriented to project visible light through the transparent tip portion and into a body cavity to provide illumination. In wireless embodiments of the finger cot camera system, the housing can contain a transmitter for wirelessly transmitting the image data captured by the camera to an external viewing system. In wireless embodiments, the camera, light source, and transmitter can receive power from a power source in the insertable camera device, such as an on-board battery.

The devices and subsystems of the external viewing system of the finger cot camera system can provide the capability to view the images captured by the camera of the insertable camera device. Wireless embodiments can include a signal receiver for wirelessly receiving the image data sent from the transmitter of the insertable camera device. The receiver, in turn, can provide the image data to an image processing system. The image processing system can manipulate the image data to a format suitable for viewing on a monitor. The processed image data can then be provided to a viewing monitor for real-time viewing of the images captured by the camera of the insertable camera device. In some cases, the processed image data from the image processing system can be optionally stored in a memory device for later viewing.

A finger cot camera system in accordance with some embodiments provided herein can be operated in the following general manner. An operator of the finger cot camera system, such as a physician, can don the insertable camera device in the normal manner for installing a finger cot on a finger. The finger cot is suitably wearable on a finger of a gloved, or an ungloved, hand. The operator can power-up the insertable camera device by activating an electrical switch on the housing or on the external viewing system. Prior to insertion, operation of the insertable camera device can be confirmed by observing that the light source is projecting visible light through the transparent tip portion. Prior to insertion, operation of the overall finger cot camera system can be confirmed by observing that the external viewing monitor can display images that are captured by the camera.

The operator can place the finger that is wearing the insertable camera device in a body cavity of the patient in the normal manner for performing a digital exam. In some cases, a probe can be used as an alternative to an operator's finger, including within orifices with a small diameter. The light source of the insertable camera device can illuminate the otherwise dark cavity. The camera can capture images from within the cavity. The image data can be transmitted to the external viewing system. The operator can view the images captured by the camera by viewing the external viewing monitor. The operator can manipulate the viewing direction of the insertable camera device within the cavity by pointing the finger in the direction desired. At the end of the procedure, the operator can remove the finger and the insertable camera device from the cavity of the patient. In some embodiments, the insertable camera device can be disposed of after a single use.

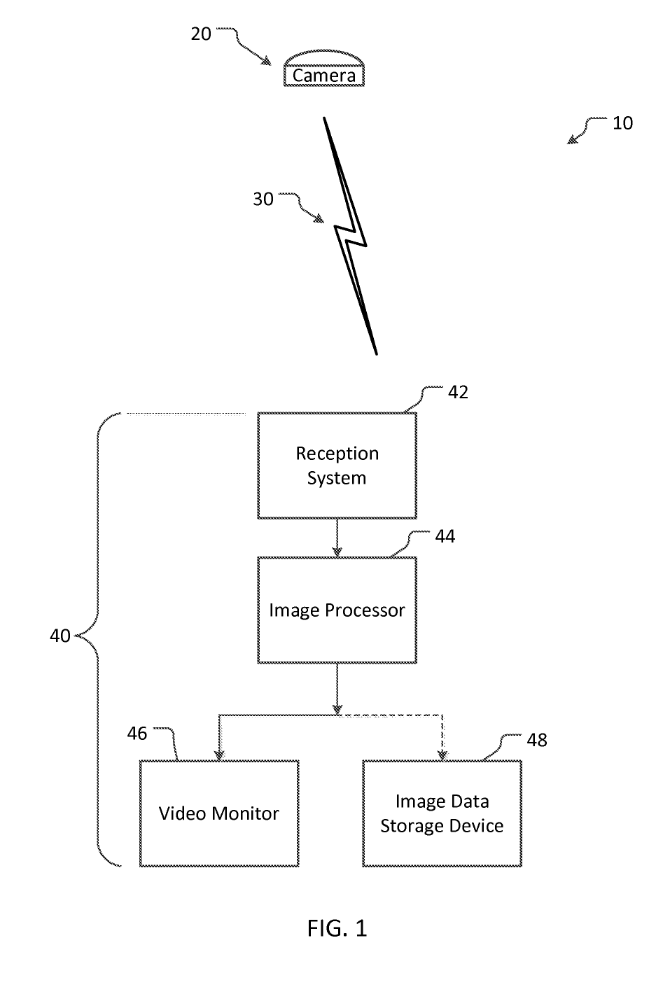

With reference to the system schematic of FIG. 1, a finger cot camera system 10 can include an insertable camera device 20, a data connection 30, and an external viewing system 40. In general, insertable camera device 20 can be used to capture images from inside an individual's body cavity. The images can be transmitted via data connection 30 to external viewing system 40. External viewing system 40 can include a viewing monitor for displaying the images to a clinician operator.

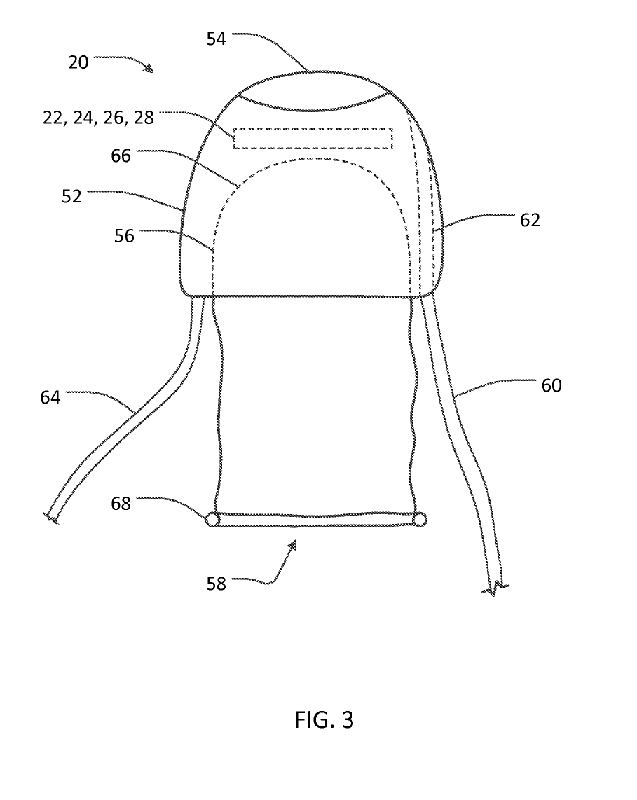

In some embodiments, insertable camera device 20 can be used to enhance the capabilities of a digital examination procedure. As described further in reference to FIG. 3 below, insertable camera device 20 can be worn on a finger of an operator as an integral part of a finger cot or surgical glove. The finger wearing insertable camera device 20 can be inserted into a cavity of a patient in the usual manner of a digital examination procedure. The digital examination procedure can be performed by a physician, veterinarian, another clinician operator, or, in some cases, by the patient themselves. Insertable camera device 20 can capture images from inside the cavity of the patient. Because insertable camera device 20 is worn on the finger of the operator, the operator can conveniently manipulate the position of insertable camera device 20 to point the camera to view particular areas of the cavity as desired by the operator. For example, if a particular area of the cavity is suspected as being abnormal, the operator can point the insertable camera device 20 in the direction of the suspected abnormality.

In some embodiments, insertable camera device 20 can be adapted for installation on a probe device. The use of a probe can, in some cases, enable viewing of portions of a body cavity that are located further within the body cavity (e.g., ear, nose, and the like) as compared to attachment of insertable camera device 20 on the finger of an operator. In some embodiments, at least the tip of the probe can be steerable to enhance the capability to manipulate insertable camera device 20 within the cavity.

Image data from insertable camera device 20 can be transmitted to external viewing system 40 via data connection 30. In some embodiments, data connection 30 can be a wireless connection. For example, data connection 30 can enable wireless communications between insertable camera device 20 and external viewing system 40 using radio frequency (RF) technologies. The radio frequency can be, for example, in the range of 900 MHz to 5.8 GHz. In some embodiments, data connection 30 can use Wi-Fi technology such as 802.11b or 802.11g for wireless data transmission. In some embodiments, data connection 30 can use microwave technology for wireless data transmission. In some embodiments, a combination of wireless technologies can be used by data connection 30.

In some cases, data connection 30 can be a wired connection, or a wired connection can be an available backup to a wireless connection. Embodiments using a wired connection can use the wire to transmit image data from insertable camera device 20 to external viewing system 40, and to transmit electrical energy to insertable camera device 20 so that insertable camera device 20 may not need an on-board battery. The wire can also serve as a retrieval mechanism in the event insertable camera device 20 becomes dislodged from the operator's finger or probe.

External viewing system 40 includes a reception system 42, an image processor 44, a viewing monitor 46, and, optionally, an image data storage device 48. External viewing system 40 is located externally to the patient. External viewing system 40 can be in data communication with insertable camera device 20 via data connection 30.

Reception system 42 can be included when data connection 30 is a wireless connection. Reception system 42 can include an antenna and circuitry for receiving a wirelessly transmitted signal containing image data from insertable camera device 20. Reception system 42 can pass the image data to image processor 44.

Image processor 44 can be used to improve the quality of the still images or video for display, for example, by making the images sharper and eliminating signal noise. In some embodiments, image processor 44 can enhance the color, brightness, or contrast of images transmitted from insertable camera device 20, prior to display on viewing monitor 46.

In some embodiments, image processor 44 can provide freeze-frame functionality for embodiments of insertable camera device 20 that include a video camera. For example, when the operator manipulates insertable camera device 20 to view a particular feature within the cavity of a patient, the operator can initiate the capture of a freeze-frame image of the feature. In that case, the frozen image would remain on viewing monitor 46 even after the operator changes the view of insertable camera device 20. In some embodiments, the frozen image would remain on viewing monitor 46 until a subsequent selection to clear the image is entered into image processor 44 by the operator.

Viewing monitor 46 can display the images captured by insertable camera device 20. Viewing monitor 46 can be a color or black and white display. Viewing monitor 46 can be positioned to provide convenient viewing by the operator. In some embodiments, viewing monitor 46 can conveniently be a hand-held device. In that case, the operator could perform the digital examination with one hand and hold the hand-held viewing monitor 46 with another hand. In some embodiments, viewing monitor 46 can be located on a mobile cart and/or attached to an articulating arm.

External viewing system 40 can optionally include image data storage device 48. Image data storage device 48 can save the images created during a body cavity examination using insertable camera device 20. The images can be saved for review at a later time, for example, by a physician or a specialist. In some embodiments, image data storage device 48 can be a hard drive data storage system. In some embodiments, image data storage device 48 can be a RAM system, a DVD, a memory stick, and the like.

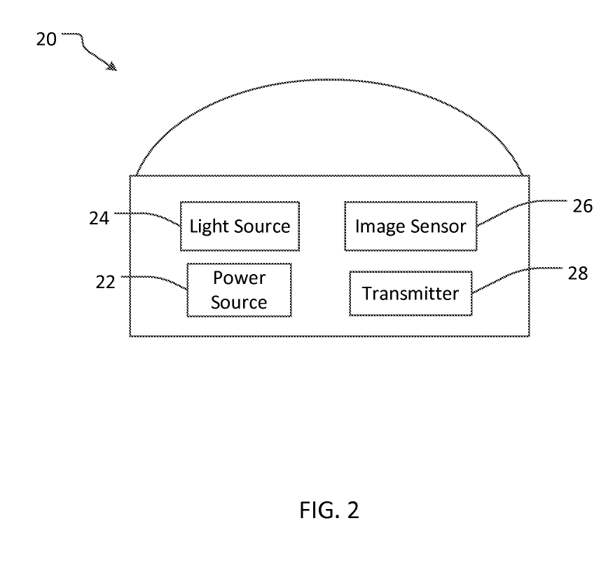

With reference to the schematic drawing of FIG. 2, the components of insertable camera device 20 can include a power source 22, a light source 24, an image sensor 26, and a transmitter 28. In some embodiments, such components can be positioned together on a single printed circuit board within insertable camera device 20. In some embodiments, some of such components can be located separately from other components while being in wired electrical communication with each other.

Power source 22 can be, in some embodiments, a battery. For example, in embodiments of finger cot camera system 10 using a wireless data connection 30, an on-board battery can comprise power source 22 without requiring external wires to insertable camera device 20. A battery power source 22 can be, for example, an alkaline, nickel-metal hydride, lithium ion, lithium polymer, or zinc oxide battery. In some embodiments, a battery life of 10-15 minutes may be acceptable. In some embodiments, a longer battery life of 15-30 minutes, or more, may be desirable.

In some embodiments, power source 22 can receive electrical power from a source that is remote to insertable camera device 20. For example, in embodiments of finger cot camera system 10 using a wired data connection 30, the electrical power can be provided to insertable camera device 20 via wire conductors that can be routed along with the wires for wired data connection 30. In such cases, battery life would not limit the time that finger cot camera system 10 can be in operation.

In some embodiments, power source 22 can include a selectable power activation switch. In such cases, the power switch can be manually activated to provide electricity from power source 22 to the other components of insertable camera device 20. In some embodiments, the power switch can be manually deactivated to de-power the components of insertable camera device 20. Power source 22 can provide electrical energy to the other components of insertable camera device 20, including, among others, light source 24.

Insertable camera device 20 can include light source 24. Light source 24 can provide a means of visual illumination within the confines of a body cavity, to enable the features within the cavity to be visualized. In some embodiments, light source 24 can be a light emitting diode (LED) component. In some embodiments, light source 24 can be another type of light source, such as an incandescent halogen lamp. In some embodiments, a lens can be included to direct or diffuse the light from light source 24 as desired.

Other additional types of energy emission sources can be included in some embodiments of insertable camera device 20. In some embodiments, other types of energy emission sources can emit wavelengths of energy that can be useful for additional diagnostic purposes. For example, in some embodiments, an energy emission source to induce autofluorescence can be included. Such energy emission sources can be useful, for example, for visualizing the presence of certain fluorescent dyes. Other real-time, on-demand endoscopic imaging techniques designed to improve visualization and tissue characteristics (e.g., vascular pattern), and diagnosis may be used such as NBI (narrow band imaging), MBI (multiband imaging), FICE (Fujinon intelligent chromoendoscopy), and the like.

Insertable camera device 20 can include image sensor 26. Image sensor 26 can be, for example, a miniaturized camera device. In some embodiments, a CMOS-based camera can be used. In some embodiments, a CCD-based camera can be used. In some embodiments, one or more optical lenses and/or filters can be included with image sensor 26. For example, the use of a lens can enable image sensor 26 to capture a wider viewing angle. In some embodiments, a viewing angle of approximately 180 degrees can be attained. Image sensor 26 can receive electrical energy from power source 22. When data connection 30 is a wired connection, the image data from image source 26 can be transmitted via wired connection 30 to image processor 44. In wireless embodiments of insertable camera device 20, image sensor 26 can output still frame or video image data signals to transmitter 28.

In embodiments using a wireless data connection 30, transmitter 28 can receive image data signals from image sensor 26. Transmitter 28 can wirelessly transmit image data using, for example, RF signal transmission technology. In some embodiments, transmitter 28 can include components such as a microprocessor, an oscillator, a modulator, amplifiers, filters, and an antenna.

In some embodiments, transmitter 28 can be a transceiver device. By using such a transceiver device, additional features can be incorporated into finger cot camera system 10. For example, control commands can be wirelessly sent from external viewing system 40 to insertable camera device 20. In some embodiments, the power source 22 can be controlled from external viewing system 40. That is, for example, insertable camera device 20 can be powered-up and powered-down remotely from external viewing system 40. In some embodiments, the intensity of the light source 24 can be remotely controlled from external viewing system 40. That is, two or more levels of visible light intensity can be selectable by the operator. In some embodiments, additional types of energy emission sources can be controlled remotely from external viewing system 40. The life of the on-board battery can be extended by only powering such energy emission sources on an as-needed basis.

With reference to FIG. 3, insertable camera device 20 can include a housing 52 fixedly coupled to a finger cot 58. Optionally, insertable camera device 20 can also include a tube 60, and a cable 64 for a wired data connection 30.

Housing 52 can comprise a biocompatible polymeric material, e.g., polycarbonate, polyvinylchloride, polytetrafluoroethylene, and the like. In some embodiments, housing 52 can be formed by injection molding. In some embodiments, housing 52 can comprise two or more molded components that can be assembled during secondary assembly operations. Secondary assembly operations can also be used to install power source 22, light source 24, image sensor 26, and transmitter 28 within housing 52. Housing 52 can include a transparent portion 54. Transparent portion 54 can be an optically clear window to enable the operation of light source 24 and image sensor 26 to illuminate and capture images external to housing 52. Transparent portion 54 can comprise, for example, polycarbonate. In some embodiments, transparent portion 54 can be insert-molded with housing 52 to integrally incorporate transparent portion 54 with housing 52.

Optionally, housing 52 can include a lumen 62. Lumen 62 can be connected to a tube 60 at a distal end of tube 60. The other (proximal) end of tube 60 can be located external to the patient where the tube 60 can be readily accessible to the operator. In some embodiments, the inner diameter of tube 60 and lumen 62 can be about 1 to about 2 mm. In some embodiments, the inner diameter of tube 60 and lumen 62 can be about 2 to about 4 mm, or larger. Tube 60 and lumen 62 can be used for various purposes. For example, in some embodiments, tube 60 and lumen 62 can be used as a channel for sample collection using a brush, forceps, swab, suction catheter, etc. In some embodiments, tube 60 and lumen 62 can be used for insufflation of the cavity with, for example, air or CO₂. In some embodiments, tube 60 and lumen 62 can be used for aspiration or fluid cleansing of the cavity to enhance visibility.

Optionally, housing 52 can have a cable 64 attached to housing 52. Cable 64 can be included, for example, when data connection 30 is a wired connection. In some embodiments, cable 64 can be coupled to tube 60 to comprise a single flexible element connected to housing 52. Cable 64 can include, for example, electrical wires for transmitting image data from insertable camera device 20 to external viewing system 40, and for providing electrical power from external viewing system 40 to insertable camera device 20.

In some cases, housing 52 can also include a finger well 56. Finger well 56 can provide a space for the operator's fingertip. In some embodiments, a power switch 66 for insertable camera device 20 can be located in finger well 56. In some embodiments, power switch 66 can be flush with the surface of finger well 56 when insertable camera device 20 is powered-up, but slightly protruding from finger well 56 when insertable camera device 20 is not powered-up. In such case, the operator can select power switch 66 by contacting power switch 66 with the operator's fingertip. Finger well 56 can also provide a surface on which to attach a finger cot 58.

Finger cot 58 can be similar to a standard finger cot except a portion of finger cot 58 is attached to housing 52. In some cases, housing 52 can be attached near the tip of finger cot 58. In some cases, housing 52 can be attached to the side or other portions of finger cot 58. The attachment of finger cot 58 to housing 52 can be accomplished by using, for example, adhesive, ultrasonic welding, insert molding, clamping, and the like. Finger cot 58 can provide a secure fit on the operator's finger to prevent slippage of insertable camera device 20 from operator's finger. For example, in some cases, a stretchable and self-adjustable elastic band 68 is included on finger cot 58. Finger cot 58 can be worn on a finger of a hand wearing a surgical glove. In some cases, housing 52 can be attached directly to a finger of a surgical glove rather than finger cot 58.

Insertable camera device 20 can be made in a range of sizes, lengths, diameters, and shapes (e.g. cylindrical, spherical, conical, and the like) to accommodate different operators and different body cavities. In some embodiments, insertable camera device 20 can be a single-use disposable item, while external viewing system 40 can be reusable.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A method for performing a digital exam on a mammal, wherein said method comprises inserting a camera within a body cavity of the mammal, wherein said camera is removably coupled to a finger of an operator performing the digital exam.

2. The method of claim 1, wherein said mammal is a human.

3. The method of claim 1, further comprising transmitting image data from said camera to an external viewing system.

4. The method of claim 3, wherein said transmitting comprises a wireless transmission.

5. The method of claim 3, wherein said transmitting comprises a wired transmission.

6. An insertable camera device for performing a visual examination within a body cavity of a mammal, wherein said device comprises a camera attached to a finger cot, wherein said device is configured to be wearable on a finger of an operator and to be inserted within the cavity by the operator wearing the device.

7. The device of claim 6, wherein said mammal is a human.

8. The insertable camera device of claim 6, wherein said device further comprises a lumen configured for passing substances or items therethrough.

9. The insertable camera device of claim 6, wherein said device further comprises a battery configured to provide electrical power to operate said device.

10. The insertable camera device of claim 6, wherein said device further comprises a wireless transmitter configured to wirelessly transmit image data captured by said camera.

11. The insertable camera device of claim 6, wherein said device further comprises a light source.

12. The insertable camera device of claim 11, wherein said light source comprises a light emitting diode.

13. A finger cot camera system configured for performing a visual examination within a body cavity of a mammal, wherein said system comprises a camera attached to a finger cot and an external viewing system, wherein said camera is configured to be wearable on a finger of an operator and to be inserted within the cavity by the operator wearing the device, and wherein said external viewing system is configured to receive and display images from said camera.

14. The finger cot camera system of claim 13, wherein said external viewing system is configured to be handheld.

15. The finger cot camera system of claim 13, wherein said mammal is a human.

16. The finger cot camera system of claim 13, wherein said camera further comprises a lumen configured for passing substances or items therethrough.

17. The finger cot camera system of claim 13, wherein said camera further comprises a battery configured to provide electrical power to operate said camera.

18. The finger cot camera system of claim 13, wherein said camera further comprises a wireless transmitter configured to wirelessly transmit image data captured by said camera to said external viewing system.

19. The finger cot camera system of claim 13, wherein said camera further comprises a light source.

20. The finger cot camera system of claim 19, wherein said light source comprises a light emitting diode.
